(19) United States
(12) Patent Application Publication
(10) Pub. No.: US 2009/0221039 A1
(43) Pub. Date: Sep. 3, 2009

(54) FUSION PROTEINS BETWEEN PLANT CELL-WALL DEGRADING ENZYMES AND A SWOLLENIN, AND THEIR USES

(75) Inventors: Eric Record, Marseille (FR); Anthony Levasseur, Aubagne (FR); Markku Soloheimo, Helsinki (FI); David Navarro, Marseille (FR); Martina Andgerg-Blomster, Fin-Kirkkonummi (FI); Frederic Monot, Nanterre (FR); Tiina Nakari-Setala, Espoo (FI); Marcel Asther, La Ciotat (FR)

Correspondence Address: YOUNG & THOMPSON, 209 Madison Street, Suite 500, ALEXANDRIA, VA, 22314, US

(73) Assignees: Institut Francias Du Petrole, Rueil-Malmaison (FR); VTT Technical Research Centre of Finland, Espoo (FI); Institute National De La Recherche Agronomique, Paris (FR); Universite De Provence, Marseille (FR)

(21) Appl. No.: 12/295,870

(22) PCT Filed: Apr. 2, 2007
(86) PCT No.: PCT/EP2007/002947

§ 371 Date: May 7, 2009

(30) Foreign Application Priority Data
Apr. 6, 2006 (EP) 06290562.5

(51) Int. Cl. C12P 21/04; C12N 9/14; C12N 9/42; C12N 1/15; C12N 15/63; A62D 3/02; C12S 3/00; C07H 21/04

(52) U.S. Cl. 435/69.7; 435/195; 435/209; 435/254.11; 435/254.3; 435/254.6; 435/320.1; 435/262.5; 435/267; 536/23.2

(57) ABSTRACT

The invention relates to fusion proteins including at least a swollenin and at least a plant cell-wall degrading enzyme, the swollenin, and plant cell-wall degrading enzyme, being recombinant proteins corresponding to native proteins in fungi, or mutated forms thereof. The invention also relates to the use of fusion proteins as defined above, for carrying out processes of plant cell-wall degradation in the frame of the preparation, from plants or vegetal by-products, of compounds of interest located in plant cell-wall, or in the frame of the bleaching of pulp and paper, or for biofuel production, or food industries.

The invention relates to the construction and overproduction of engineered multifunctional fusion proteins between at least a swollenin and at least a plant cell-wall degrading enzyme, and to their uses as improved enzymatic tools for valorisation of agricultural by-products.

### BACKGROUND OF THE INVENTION

The plant cell wall has developed a complex architecture with an intrinsic composition of diverse carbohydrates in order to protect the cell from microbial attacks. As the consequence, plant cell wall-degrading micro-organisms have designed several enzymatic systems to break down the plant biomass and to finally assimilate the sugar substrates. Among bacterial and fungal micro-organisms, modular enzymes are found containing Carbohydrate-Binding Modules (CBMs) that assist enzymes for substrate targeting. Recently, a new kind of proteins, involved in the plant cell wall disruption, was identified in Trichoderma reesei and named swollenin (Saloheimo M. et al. 2002). This protein presents high similarity with plant expansins that breakdown hydrogen bounds between cellulose microfibrils or cellulose and other cell wall polymers (Cosgrove 2000). Indeed, plant expansins are thought to play a role in the cell wall extension and are considered as a key endogenous regulator for the cell wall growth of the plant (Li Y et al. 2003). In contrast to plant expansins, the swollenin has a bi-modular structure composed of a CBM connected by a linker region to the plant expansin homologous domain. This modular structure is typical of fungal cellulases and some hemicellulases that present a CBM to target the enzymatic module. In the specific case of the swollenin, there is no associated hydrolytic activity but an expansin module with cell wall disruption capacity. In parallel, micro-organisms cell has developed free systems that do not possess a CBM module but are secreted in large quantities in the extracellular medium. These kinds of enzymes are found among cellulases, hemicellulases and pectinases. Genetic engineering studies have focused on the improvement of free enzymes by associating a CBM module to target enzymes to a specific plant substrate such as cellulose (Ito et al. 2004; Limón et al. 2004). In the first case, Ito et al. demonstrated that the hydrolytic activity of a T. reesei endoglucanase was increased with the number of CBM added to the enzyme. In the second work, a COM module was genetically fused to a non-cellulase enzyme, the Trichoderma harzanium chitinase, and results showed that both chitinase and antifungal activities increased with increasing binding capacity to cellulose. This performance gain is of great interest for industrial applications where the plant cell wall degradation is a key-point, i.e. in the biofuel and in the pulp and paper sectors.

Recently, the inventors became interested in cinnamoyl esterases that are able to hydrolyse different kinds of sugar ester-linked hydroxycinnamic acids. These enzymes were classified on the basis of substrate specificity and primary sequence identity (Crepin et al. 2003). The first cinnamoyl esterases to be fully characterized belong to Aspergillus niger. The feruloyl esterase (FAEIII, type A) was described to be preferentially active against methyl ester of ferulic and sinapic acids (Faulds and Williamson 1994), while the cinnamoyl esterase showed a preference for the methyl ester of caffeic and p-coumaric acids (Kroon et al. 1996). Both encoding genes were cloned and characterized. They were overexpressed in Pichia pastoris and A. niger to yield sufficient quantities of recombinant proteins and enable their utilisation in industrial applications (Juge et al. 2001; Record et al. 2003, Levasseur et al. 2003). The feruloyl esterase was evaluated for wheat straw and flax pulp bleaching and demonstrated to improve, in combination with a laccase treatment, the decrease of the final lignin content (Record et al. 2003; Sigoillot et al; 2005). Indeed, the feruloyl esterase is known to hydrolyse feruloylated oligosaccharides but also diferulate cross-links found in hemicellulose and pectin (Williamson et al. 1998; Saulnier and Thibault 1999), facilitating the access of other ligno-cellulolytic enzymes.

The aim of the present work is to develop new enzymatic tools to degrade plant biomass or to biotransform plant cell wall components. Two strategies were developed in parallel. In a previous work (Levasseur et al. 2004), the goal was to design a new kind of fungal enzyme fused to a bacterial dockerin and therefore able to be incorporated in cellulosome from Clostridium thermocellium. Indeed, bacterial cellulosome is a very effective system for increasing the synergistic effect of enzymes (Ciruela et al. 1998, Fierobe et al. 2002). In an alternative way, chimerical enzymes associating two enzymes were shown to be very effective to degrade the plant biomass and especially if a CBM module was integrated in the enzymatic complex (Levasseur et al 2005). In other works, the fusion of CBM modules to enzymatic partners was reported to be a good way to improve the efficiency of the enzymatic partner by assisting the enzyme targeting to the substrate and increasing the local concentration of the enzymes (Cages et al. 1997, Boraston et al. 2004).) In addition, only a few CBMs were reported to mediate non-catalytic disruption effect of the crystalline structure of the cellulose pin et al. 1994, Gao et al. 2001).

In the present invention, the inventors describe for the first time the association of a swollenin to a plant cell wall-degrading enzyme, such as the feruloyl esterase used as an enzyme model, by using a genetic fusion of the both corresponding genes.

### SUMMARY OF THE INVENTION

The present invention relies on the demonstration of the effect on enzymatic efficiency, related to the physical association in a single chimerical protein, of plant cell-wall degrading enzymes and swollenin, when compared to the use of the free plant cell-wall degrading enzymes.

Thus the main goal of the present invention is to provide new fusion proteins between swollenin and plant cell-wall degrading enzymes.

Another goal of the present invention is to provide a new process for the preparation of compounds of interest linked to the walls of plant cells, by applying said fusion proteins to plants, and advantageously to agricultural by-products, as substrates.

### BRIEF DESCRIPTION OF THE DRAWINGS

FIG. 1 represents the Feruloyl esterase (FAEA) production in Trichoderma reesei. Feruloyl esterase activity was measured in the extracellular medium obtained from the best FAEA transformants of T. reesei Rut-C () and CL847 (▪). Methyl ferulate was used as substrate for activity tests.

FIG. 2 illustrates Western blot analysis and copy number of integrated cassettes in the genome of T. reesei. Antibodies raised against FAEA were used for immunodetection of FAEA and SWOI-FAEA transformants from the total extracellular media, Lane 1 and 2: Rut-C30 tansformants producing FAEA and SWOI-FAEA, respectively. Lane 3 and 4: CL847 transformants producing FAEA and SWOI-FAEA, respectively. Copy number of expression cassettes was estimated by Southern blot analysis. The wild-type Aspergillus niger strain BRFM was used as control containing one fae A gene copy. Sd: molecular weight standards.

FIG. 3 represents SDS-PAGE gel of extracellular and purified proteins of Trichoderma reesei. Lane 1: non-transformed T. reesei CL847 strain. Lane 2 and 3: Total extracellular media of T. reesei CL847 strain transformed by the expression cassettes for FAEA or SWOI-FAEA production, respectively. Lanes 4 and 5: purified FAEA and SWOI-FAEA. Sd: molecular weight standards.

FIG. 4 shows the temperature stability of FAEA and SWOI-FAEA obtained from Trichoderma reesei strain 847. Activity of the purified protein FAEA (♦) and SWOI-FAEA (▪) after 60 min of incubation at the indicated temperature is represented. Methyl ferulate was used as substrate for activity tests.

FIG. 5 illustrates the ferulic acid release by using FAEA or SWOI-FAEA of Trichoderma reesei CL847. Wheat bran was used as substrate and ferulic acid release was determined by HPLC after 4 h (white bars), 16 h (grey bars) and 24 h (black bars) of hydrolysis. Activities were expressed as the percentage of the total amount of ferulic acid in wheat bran. R: reference containing only the buffer; S: extracellular medium of the non transformed strain; C: control as the feruloyl esterase from Aspergillus niger; F: feruloyl esterase (FAEA) from T reesei, S: swollenin (SWOI) from T. reesei; S—F fusion protein (SWOI-AEA) from T. reesei.

### DETAILED DESCRIPTION OF THE INVENTION

The invention relates to fusion proteins comprising:
at least a swollenin, i.e. a protein containing a carbohydrate-binding-molecule (CBM) domain which targets the cellulose of plants, and an expansin domain which breakdowns hydrogen bounds between cellulose microfibrils,
and at least a plant cell-wall degrading enzyme, said enzyme being such that it contains a CBM domain or not, provided that when it contains a CBM this latter may be deleted if necessary,

said swollenin, and plant cell-wall degrading enzyme, being recombinant proteins corresponding to native proteins in fungi, or mutated forms thereof.

The expression “plant cell-wall degrading enzymes” refers to enzymes that are able to perform the digestion of the cell-wall components, such as cellulose, hemicellulose and lignin. The plant cell-wall degrading enzymes in said fusion proteins are identical, or different from each other.

The expression “Carbohydrate-binding-molecule” refers to a molecule with affinity to cellulose that targets its associated enzyme to the cellulose,

The invention relates more particularly to fusion proteins as defined above, wherein the swollenin corresponds to native proteins, or mutated forms thereof, from fungi chosen among ascomycetes, such as
Trichoderma strains, and more particularly Trichoderma reesei, or
Aspergillus strains, and more particularly Aspergillus fumigatus.

The invention concerns more particularly fusion proteins as defined above, wherein the swollenin corresponds to native enzymes, or mutated forms thereof, from Trichoderma strains, such as Trichoderma reesei.

The invention more particularly relates to fusion proteins as defined above, wherein the swollenin is the protein of Trichoderma reesei, represented by:
SEQ ID NO: 2 in its pre-protein state, i.e. containing the signal peptide SEQ ID NO 102 of the following 18 aminoacids: MAGKLILVALASLVSLSI,
or by SEQ ID NO: 4 in its mature state, i.e. without the above-mentioned signal peptide.

The invention more particularly concerns fusion proteins as defined above, wherein the swollenin corresponds to native enzymes, or mutated forms thereof, from Aspergillus strains, such as Aspergillus fumigatus.

The invention more particularly relates to fusion proteins as defined above, wherein the swollenin is the protein of Aspergillus fumigatus, represented by:
SEQ ID NO: 6 in its pre-protein state, i.e. containing the signal peptide SEQ ID NO: 104 of the following 17 aminoacids: MTLLFGTFLARLAVAAA,
or by SEQ ID NO: 8 in its mature state, i.e. without the above-mentioned signal peptide.

The invention more particularly concerns fusion proteins as defined above, wherein the plant cell-wall degrading enzymes are chosen among enzymes able to hydrolyze cellulose, hemicellulose, and degrade lignin.

The invention more particularly relates to fusion proteins as defined above, wherein the plant cell-wall degrading enzymes are hydrolases chosen, among:
cellulases, such as endoglucanases, exoglucanases such as cellobiohydrolases, or β-glucosidases,
hemicellulases, such as xylanases,
ligninases able to degrade lignins, such as laccases, manganese peroxidases, lignin peroxidases, versatile peroxidases, or accessory enzymes such as cellobiose deshydrogenases, and aryl alcohol oxidases,
cinnamoyl ester hydrolases able to release cinnamic acids such as acids ferulic acids and to hydrolyse diferulic acid cross-links between hemicellulose chains, such as feruloyl esterases, cinnamoyl esterases, and chlorogenic acid hydrolases.

The invention more particularly concerns fusion proteins as defined above, wherein the plant cell-wall degrading enzymes are chosen among feruloyl esterases, cellobiohydrolases with or without their CBM domains, endoglucanases with or without their CBM domains, xylanases, and laccases.

The invention more particularly relates to fusion proteins as defined above, wherein the plant cell-wall degrading enzymes correspond to native enzymes, or mutated forms thereof, from fungi chosen among:
ascomycetes, such as
   Aspergillus strains, and more particularly Aspergillus niger,
   Trichoderma strains, and more particularly Trichoderma reesei,
   Magnaporthe strains, and more particularly Magnaporthe grisea,
basidiomycetes, such as Pycnoporus, Halocyphina, or Phanerochaete strains, and more particularly Pycnoporus cinnabarinus, Pycnoporus sanguineus, or Halocyphina villosa, or Phanerochaete chrysosporium.

The invention more particularly concerns fusion proteins as defined above, wherein the plant cell-wall degrading enzymes correspond to native enzymes, or mutated forms thereof, from Aspergillus strains, such as Aspergillus niger.

The invention more particularly relates to fusion proteins as defined above, wherein at least one of the plant cell-wall degrading enzymes is a feruloyl esterase, such as the one chosen among:
the feruloyl esterase A of A. niger represented by SEQ ID NO: 10,
or the feruloyl esterase B of A. niger represented by SEQ ID NO: 12.

The invention more particularly concerns fusion proteins as defined above, wherein at least one of the plant cell-wall degrading enzymes is a xylanase such as the xylanase B of A. niger represented by SEQ ID NO: 14.

The invention more particularly relates to fusion proteins as defined above, wherein the plant cell-wall degrading enzymes correspond to native enzymes, or mutated forms thereof, from Trichoderma strains, such as Trichoderma reesei.

The invention more particularly concerns fusion proteins as defined above, wherein at least one of the plant cell-wall degrading enzymes is a cellobiohydrolase, such as the one chosen among:
the cellobiohydrolase I of T. reesei, and represented by SEQ ID NO: 16,
the cellobiohydrolase I of T. reesei, wherein the CBM domain has been deleted, and represented by SEQ ID NO:18,
the cellobiohydrolase II of T. reesei, and represented by SEQ ID NO: 20,
the cellobiohydrolase II of T. reesei, wherein the CBM domain has been deleted, and represented by SEQ ID NO: 22.

The invention more particularly relates to fusion proteins as defined above, wherein at least one of the plant cell-wall degrading enzymes is an endoglucanase, such as the one chosen among:
the endoglucanase I of T. reesei, and represented by SEQ ID NO: 24,
the endoglucanase I of T. reesei, wherein the CBM domain has been deleted, and represented by SEQ ID NO: 26

The invention more particularly concerns fusion proteins as defined above, comprising linkers between at least two of the proteins comprised in said fusion proteins, said linkers being polypeptides from 10 to 100 aminoacids, advantageously of about 50 aminoacids.

The invention more particularly relates to fusion proteins as defined above, wherein a linker is included between each protein comprised in said fusion proteins.

The invention also more particularly relates to fusion proteins as defined above, wherein the linker is a hyperglycosylated polypeptide such as the sequence represented by SEQ ID NO: 28, present in the cellobiohydrolase B of A. niger.

The invention more particularly concerns fusion proteins as defined above, chosen among the fusion proteins of the swollenin of Trichoderma reesei represented by SEQ ID NO: 4, with:
the feruloyl esterase A of A. niger represented by SEQ ID NO:10, said fusion protein being represented by SEQ ID NO: 30,
the feruloyl esterase A of A. niger represented by SEQ ID NO:10, said fusion protein comprising the sequence represented by SEQ ID NO: 28 as a hyperglycosylated linker between SEQ ID NO: 4 and SEQ ID NO: 10, and being represented by SEQ ID NO: 32,
the feruloyl esterase B of A. niger represented by SEQ ID NO: 12, said fusion protein being represented by SEQ ID NO: 34
the feruloyl esterase B of A. niger represented by SEQ ID NO: 12, said fusion protein comprising the sequence represented by SEQ ID NO: 28 as a hyperglycosylated linker between or SEQ ID NO: 4 and SEQ ID NO: 12, and being represented by SEQ ID NO: 36,
the-xylanase B of A. niger represented by SEQ ID NO: 14, said fusion protein being represented by SEQ ID NO: 38,
the xylanase B of A. niger represented by SEQ ID NO: 14, said fusion protein comprising the sequence represented by SEQ ID NO: 28 as a hyperglycosylated linker between SEQ ID NO: 4 and SEQ ID NO: 14, and being represented by SEQ ID NOV 40,
the cellobiohydrolase I of T. reesei represented by SEQ ID NO:16, said fusion protein being represented by SEQ ID NO: 42,
the cellobiohydrolase I of T. reesei represented by SEQ ID NO:16, said fusion protein comprising the sequence represented by SEQ ID NO: 28 as a hyperglycosylated linker between SEQ ID NO: 4 and SEQ ID NO: 16, and being represented by SEQ ID NO: 44,
the cellobiohydrolase I of T. reesei without its endogenous CBM represented by SEQ ID NO: 18, said fusion protein being represented by SEQ ID NO: 46,
the cellobiohydrolase I of T. reesei without its endogenous CBM represented by SEQ ID NO:18, said fusion protein comprising the sequence represented by SEQ ID NO: 28 as a hyperglycosylated linker between SEQ ID NO: 4 and SEQ ID NO: 18, and being represented by SEQ ID NO:48,

the cellobiohydrolase II of T. reesei by SEQ ID NO: 20, said fusion protein being represented by SEQ ID NO: 50,
the cellobiohydrolase II of T. reesei represented by SEQ ID NO: 20, said fusion protein comprising the sequence represented by SEQ ID NO: 28 as a hyperglycosylated linker between SEQ ID NO: 4 and SEQ ID NO: 20, and being represented by SEQ ID NO: 52,
the cellobiohydrolase II of T. reesei without its endogenous CBM represented by SEQ ID NO: 22, said fusion protein being represented by SEQ ID NO: 54,
the cellobiohydrolase II of T. reesei without its endogenous CBM represented by SEQ ID NO: 22, said fusion protein comprising the sequence represented by SEQ ID NO: 28 as a hyperglycosylated linker between SEQ ID NO: 4 and SEQ ID NO: 22, and being represented by SEQ ID NO: 56,
the endoglucanase I of T. reesei represented by SEQ ID NO: 24, said fusion protein being represented by SEQ ID NO: 58,
the endoglucanase I of T. reesei represented by SEQ ID NO: 24, said fusion protein comprising the sequence represented by SEQ ID NO: 28 as a hyperglycosylated linker between SEQ ID NO: 4 and SEQ ID NO: 24, and being represented by SEQ ID NO: 60,
the endoglucanase I of T. reesei without its endogenous CBM represented by SEQ ID NO: 26, said fusion protein being represented by SEQ ID NO: 62,
the endoglucanase I of T. reesei without its endogenous CBM represented by SEQ ID NO: 26, said fusion protein comprising the sequence represented by SEQ ID NO: 28 as a hyperglycosylated linker between SEQ ID NO: 4 and SEQ ID NO: 26, and being represented by SEQ ID NO: 64.

The invention more particularly relates to fusion proteins as defined above, chosen among the fusion proteins of the swollenin of Aspergillus fumigatus represented by SEQ ID NO: 8, with
the feruloyl esterase A of A niger represented by SEQ ID NO: 10, said fusion protein being represented by SEQ ID NO: 66,
the feruloyl esterase A of A. niger represented by SEQ ID NO: 10, said fusion protein comprising the sequence represented by SEQ ID NO: 28 as a hyperglycosylated linker between SEQ ID NO: 8 and SEQ ID NO:10, and being represented by SEQ ID NO: 68,
the feruloyl esterase B of A. niger represented by SEQ ID NO:12, said fusion protein being represented by SEQ ID NO: 70,
the feruloyl esterase B of A. niger represented by SEQ ID NO: 12, said fusion protein comprising the sequence represented by SEQ ID NO: 28 as a hyperglycosylated linker between or SEQ ID NO: 8 and SEQ ID NO:12, and being represented by SEQ ID NO: 72,
the-xylanase B of A. niger represented by SEQ ID NO: 14, said fusion protein being represented by SEQ ID NO: 74,
the xylanase B of A. niger represented by SEQ ID NO: 14, said fusion protein comprising the sequence represented by SEQ ID NO: 28 as a hyperglycosylated linker between SEQ ID NO: 8 and SEQ ID NO: 14, and being represented by SEQ ID NO: 76,
the cellobiohydrolase I of T. reesei represented by SEQ ID NO: 16, said fusion protein being represented by SEQ ID NO: 78,
the cellobiohydrolase I of T. reesei represented by SEQ ID NO: 16, said fusion protein comprising the sequence represented by SEQ ID NO: 28 as a hyperglycosylated linker between SEQ ID NO: 8 and SEQ ID NO:16, and being represented by SEQ ID NO: 80,
the cellobiohydrolase I of T. reesei without its endogenous COM represented by SEQ ID NO: 18, said fusion protein being represented by SEQ ID NO: 82,
the cellobiohydrolase I of T. reesei without its endogenous CBM represented by SEQ ID NO: 18, said fusion protein comprising the sequence represented by SEQ ID NO: 28 as a hyperglycosylated linker between SEQ ID NO: 8 and SEQ ID NO:18, and being represented by SEQ ID NO: 84,
the cellobiohydrolase II of T. reesei by SEQ ID NO: 20, said fusion protein being represented by SEQ ID NO: 86,
the cellobiohydrolase II of T. reesei represented by SEQ ID NO: 20, said fusion protein comprising the sequence represented by SEQ ID NO: 28 as a hyperglycosylated linker between SEQ ID NO: 8 and SEQ ID NO: 20, and being represented by SEQ ID NO: 88,
the cellobiohydrolase II of T. reesei without its endogenous CBM represented by SEQ ID NO: 22, said fusion protein being represented by SEQ ID NO: 90,
the cellobiohydrolase II of T. reesei without its endogenous CBM represented by SEQ ID NO: 22, said fusion protein comprising the sequence represented by SEQ ID NO 28 as a hyperglycosylated linker between SEQ ID NO: 8 and SEQ ID NO: 22, and being represented by SEQ ID NO: 92,
the endoglucanase I of T. reesei represented by SEQ ID NO: 24, said fusion protein being represented by SEQ ID NO: 94,
the endoglucanase I of T. reesei represented by SEQ ID NO: 24, said fusion protein comprising the sequence represented by SEQ ID NO: 28 as a hyperglycosylated linker between SEQ ID NO: 8 and SEQ ID NO: 24, and being represented by SEQ ID NO: 96,
the endoglucanase I of T. reesei without its endogenous CBM represented by SEQ ID NO: 26, said fusion protein being represented by SEQ ID NO: 98,
the endoglucanase I of T. reesei without its endogenous CBM represented by SEQ ID NO: 26, said fusion protein comprising the sequence represented by SEQ ID NO: 28 as a hyperglycosylated linker between SEQ ID NO: 4 and SEQ ID NO: 26, and being represented by SEQ ID NO: 100.

The invention also concerns nucleic acids encoding a fusion protein as defined above, and more particularly nucleic acids chosen among SEQ ID NO: 29 to 99 encoding SEQ ID NO: 30 to 100, said nucleic acids optionally beginning with the sequence SEQ ID NO: 101 or 103 encoding respectively the signal peptides SEQ ID NO: 102 or 104 mentioned above located upstream from the aminoacids of SEQ ID NO: 30 to 100.

The invention also relates to vectors transformed with a nucleic acid as defined above.

The invention also concerns host cells transformed with a nucleic acid as defined above, using a vector as defined above.

The invention also relates to transformed host cells as defined above, chosen among fungi cells, such as the fungi as defined above, and more particularly A. niger, A. fumigatus, Trichoderma reesei, or Pycnoporus cinnabarinus.

The invention more particularly concerns a process for the preparation of fusion proteins as defined above, comprising the culture in vitro of host cells as defined above, the recovery, and if necessary, the purification of the fusion proteins produced by said host cells in culture.

The invention more particularly relates to the use of fusion proteins as defined above, for carrying out processes of plant cell-wall degradation in the frame of the preparation, from plants or vegetal by-products, of compounds of interest located in plant cell-wall, or in the frame of the bleaching of pulp and paper, or for biofuel production, or food industries.

The invention more particularly concerns the use as defined above for carrying out processes of plant cell-wall degradation in the frame of the preparation of the following compounds of interest:
bioethanol,
anti-oxidants, such as ferulic acid, or caffeic acid that are cinnamic acids and hydroxytyrosol or gallic acid
flavours, such as vanillin or p-hydroxybenzaldehyde obtained from the biotransformation of the ferulic or the p-coumaric acid, respectively.

The invention also relates to the use as defined above, wherein said fusion proteins are directly added to the plants or vegetal by-products as substrates for their hydrolysis.

The invention also relates to the use as defined above, wherein host cells transformed with nucleic acids encoding said fusion proteins, such as the fungi mentioned above, and more particularly A. niger and Pycnoporus cinnabarinus, are contacted with said plants or vegetal by-products as substrates for their hydrolysis.

The invention more particularly relates to a process of plant cell-wall degradation in the frame of the preparation, from plants or vegetal by-products, of compounds of interest located in plant cell-wall, characterized in that it comprises the following steps
the enzymatic treatment of plants or vegetal by-products or industrial waste, with fusion proteins as defined above, or with transformed cells as defined above,
optionally, the physical treatment of plants or vegetal by-products by steam explosion in combination with the action of fusion proteins,
optionally, the biotransformation with appropriate microorganisms or enzymes of the compounds contained in the cell walls and released from these latter during the above enzymatic treatment,
the recovery, and if necessary, the purification, of the compound of interest released from the cell walls during the above enzymatic treatment or obtained during the above biotransformation step.

Preferably, plants treated with fusion proteins in the process according to the invention are chosen among sugar beet, wheat, maize, rice, or all the trees used for paper industries.

Preferably, vegetal by-products or industrial waste treated wish fusion proteins in the process according to the invention are chosen among wheat straw, maize bran, wheat bran, rice bran, apple marc, coffee mare, coffee by-products and olive mill wastewater.

The invention more particularly concerns a process as defined above for the preparation of anti-oxidants, such as cinnamic acids, and more particularly ferulic acid, as compounds of interest, said process comprising:
the treatment of plants or vegetal by-products with fusion proteins as defined above comprising one of the swollenin mentioned above and at least one of the following cell-wall degrading enzymes: feruloyl esterases such feruloyl esterase A and feruloyl esterase B xylanases such as xylanase B, such as defined above,
the recovery, and if necessary, the purification, of the anti-oxidants released from the cell walls of said plants or vegetal by-products.

Advantageously, in the frame of the preparation of anti-oxidants, such as ferulic acid, plants treated with fusion proteins defined above are chosen among the following: sugar beet wheat, maize, rice, or vegetal by-products or industrial waste treated with fusion proteins defined above are chosen among the following: wheat straw, maize bran, wheat bran, rice bran, apple marc, coffee marc, coffee by-products, olive mill wastewater.

The invention also relates to a process as defined above for the preparation of flavours as compounds of interest, said process comprising:
the treatment of plants or vegetal by-products with the fusion proteins as defined above, used in the frame of the preparation of anti-oxidants as defined above,
the biotransformation of the compounds released from the cell walls during the preceding step by contacting said compounds with non defined enzymes produced by microorganisms chosen among ascomycetes or basidiomycetes such as A. niger or P. cinnabarinus, respectively,
the recovery, and if necessary, the purification, of the flavours obtained at the preceding step of biotransformation.

The invention more particularly relates to a process as defined above, for the preparation of vanillin as a flavour of interest, wherein the fusion protein used is chosen among those used for the preparation of ferulic acid as defined above, and the biotransformation step is carried out by contacting the ferulic acid released from the cell walls with non defined enzymes produced by ascomycetes or basidiomycetes such as A. niger or P. cinnabarinus, respectively.

Advantageously, plants and vegetal by-products or industrial waste used in the frame of the preparation of flavours, such as vanillin, are chosen among those mentioned above for the preparation of anti-oxidants.

The invention also relates to a process as defined above, for the preparation of bioethanol as a compound of interest, said process comprising:
the treatment of plants or vegetal by-products with fusion proteins as defined above comprising one of the swollenin mentioned above and at least one of the following cell-wall degrading enzymes: feruloyl esterases such feruloyl esterase A and feruloyl esterase B, xylanases such as xylanase B, cellulases such as endoglucanase I, cellobiohydrolase I and cellobiohydrolase II, such as defined above, said treatment being advantageously combined with a physical treatment of said plants or vegetal by-products,
the biotransformation of the treated plants or vegetal by-products obtained from the preceding step to fermentescible sugars, by using fusion proteins described above or with a transformed fungus secreting said fusion proteins, in combination with enzymes chosen among cellulases, hemicellulases or esterases, or microorganisms chosen among ascomycetes such as A. niger or Trichoderma reesei,
the biotranformation of the fermentescible sugars to bioethanol by yeast.

Advantageously, plants and vegetal by-products or industrial waste used in the frame of the preparation of bioethanol are chosen among the following: wood, annual plants, or agricultural by-products.

The invention also relates to a process for the bleaching of pulp and paper, said process comprising:
the chemical and physical treatment of plants or vegetal by-products in combination with fusion proteins as defined above comprising one of the swollenin mentioned above and at least one of the following cell-wall degrading enzymes: feruloyl esterases such feruloyl esterase A and feruloyl esterase B, xylanases such as xylanase B, ligninases such as laccases, manganese peroxidases, lignin peroxidases, versatile peroxidases, or accessory enzymes such as cellobiose deshydrogenases, and aryl alcohol oxidases, such as defined above,
optionally, the biopulping of the treated plants or vegetal by-products obtained at the preceding step, with a transformed fungus, such as P. cinnabarinus, T. reesei or A. niger, secreting fusion proteins as defined above comprising one of the swollenin mentioned above and at least one of The following cell-wall degrading enzymes: feruloyl esterases such feruloyl esterase A and feruloyl esterase B, xylanases such as xylanase B, ligninases able to degrade lignins, such as laccases, manganese peroxidases, lignin peroxidases, versatile peroxidases, or accessory enzymes such as cellobiose deshydrogenases, and aryl alcohol oxidases, as defined above,
the biobleaching of the treated plants or vegetal by-products obtained at the preceding step with fusion proteins as defined above comprising one of the swollenin mentioned above and at least one of the following cell-wall degrading enzymes: feruloyl esterases such feruloyl esterase A and feruloyl esterase B, xylanases such as xylanase B, ligninases able to degrade lignins, such as laccases, manganese peroxidases, lignin peroxidases, versatile peroxidases, or accessory enzymes such as cellobiose deshydrogenases, and aryl alcohol oxidases, such as defined above.

The invention is further illustrated with the detailed description which follows of the preparation and properties of the fusion protein between a swollenin and a plant cell wall-degrading enzyme, such as the feruloyl esterase.

Briefly, the action of an expansin-like protein was evaluated, in physical combination with the feruloyl esterase, for the release of ferulic acids, which are high value compounds derived from agricultural products. This hydroxycinnamic acid is an attractive aromatic acid, known as antioxidant and flavor precursor, in the food and pharmaceutical sectors. The recombinant enzyme was produced in T. reesei, know to be a very efficient host, to secrete large amount of extracellular proteins of industrial interest. The new recombinant enzyme was characterized and purified to be tested on a natural substrate. Finally, the recombinant strain producing the multi-modular enzyme was compared to the parental strain to evaluate the strain capacity for the ferulic acid release.

The aim of the present work was to study the effect of the association of a new category of protein, the swollenin from T. reesei (Saloheimo et al. 2002), which is involved in the disruption of the cell-wall structure, to a catalytic domain. For the enzymatic partner, a free accessory enzyme, the feruloyl esterase from A. niger, was selected as the model. Unlike standard CBM modules, the fungal swollenin is composed of two different domains, one being responsible of the substrate targeting and identified as a CBM. The second domain presents a strong similarity to plant expansins which were proposed to disrupt hydrogen bonding between cellulose microfibrils without having hydrolytic activity (Cosgrove 2000, Li et al. et al. 2003). The swollenin gene was expressed in yeast and in A. niger (Saloheimo et al. 2002) and activity assays were analysed on cotton fibres, filter papers and cell walls of the Valonia alga. T. reesei swollenin was demonstrated to modify the structure of cellulose fibres without detectable amounts of reducing sugars. In addition, the effect of the swollenin was more mainly attributed to the expansin domain and especially for the cellulose from cotton fibres and paper filters. As a conclusion, the swollenin is though to be a good candidate to represent the “swelling factor”, C1, as a non hydrolytic component necessary to make the substrate more accessible for hydrolytic components, Cx (Reese et al. 1950).

The biotechnological potential of such a protein is very attractive in the framework of plant biomass valorisation and the effect of the physical grafting of the swollenin to the feruloyl esterase for the release of ferutic acid was studied. Thus, this work represents the first work of the association of three different and complementary domains in a single enzymatic tool for an integrating action of targeting, disruption and hydrolysis. The production of the chimerical protein was achieved in two T. reesei industrial strains, RutC30 and CL847, in order to compare the production capacity of both strains. T. reesei is a well-known filamentous fungus used by the industrial sector for its outstanding capacity to produce cellulases (Montenecourt and Eveleigh 1979, Durand et al. 1988), and is a strain of reference to produce new enzymes at the industrial level. In parallel, the heterologous production of the FAEA alone was performed to be used as a control in our application trials.

In order to evaluate the effect generated by the physical proximity of both partners, SWO (SEQ ID NO: 4) was fused upstream the FAEA (SEQ ID NO: 10) without linker peptide. Therefore, SWOI was used as a carrier protein to facilitate the secretion of the heterologous FAEA. For the FAEA production, the signal peptide of the FAEA was maintained to target the secretion of the protein. The recombinant proteins, FAEA and SWOI-FAEA (SEQ ID NO: 30), were successfully produced by both strains of T. reesei. Concerning the FAEA, the CL 847 strain was shown to produce higher yields than compared to the Rut-C30 strain, i.e. 70 against 30 mg l⁻¹, while for the SWOI-FAEA protein, production reached the same level of 25 mg l⁻¹ for both strains.

The efficiency of the chimerical SWOI-FAEA protein was tested for the ferulc acid release using destarched wheat bran as substrate. In these application trials, the substrate was not pretreated by the temperature, as the disruption and swelling properties of swollenin should be a specific indicator of the action of the protein on the substrate. Ferulic acid was released with similar amounts using FAEA obtained from A. niger (Record et al. 2003) or T. reesei. This result confirms that both proteins have the same properties even if they are produced by two different host strains. If the free swollenin was added to the FAEA no further release was observed. On the other hand, a 50% increase of ferulic acid release was noticed with the SWOI-FAEA as compared to the action of the corresponding free modules. In addition, the T. reesei strain producing the chimerical SWOI-FAEA protein was evaluated to estimate the capacity of the transformed strain for the release of the ferulic acid. Using the concentrated extracellular medium of the T. reesei CL847 for a short period of incubation of 4 h, 45% of the total ferulic acid was obtained, corresponding to 1.8 g of ferulic acid by kg of wheat bran. As a conclusion, our tests of application have demonstrated tat SWOI-FAEA is more efficient than compared to the free module SWOI and FAEA for the ferulic acid release. The positive effect could be the result of the substrate targeting of the protein due to the endogenous CBM of SWOI. Thus, the CBM of SWOI could increase the local concentration of the enzyme to the proximity of the substrate and increase the final yields of hydrolysis. In addition, the efficiency of the chimerical protein could be improved by the particular mobility of SWOI expansin module (Cosgrove et al. 2000). Indeed, the expansin module is supposed to facilitate the lateral diffusion of the FAEA along the surface of the cellulose microfibrils, and at the same time to disrupt the cell wall structure, both actions being synergic for the final release of the ferulic acid. Actually, the swollenin partner of the chimerical enzyme should facilitate the access of the catalytic module by increasing the spectra of action of enzyme to the less accessible area.

This study demonstrates for the first time the positive effect of the physical proximity of an accessory enzyme to a protein involved in the cell wall disruption. Therefore, these enzymatic tools represent a non-polluting alternative and cost-reducing process to existing biotechnological process for the biotransformation of agricultural products. For instance, such chimerical enzymes can be used in the pulp and paper and bioethanol production sectors with other partner combinations depending on the biotechnological applications.

### EXAMPLES

### Materials and Methods

Strains

Echerichia coli JM 109 (Promega, Charbonnières, France) was used for construction and propagation of vectors. Trichoderma reesei strain Rut-C30 (Montenecourt and Eveleigh 1979) and CL847 (Durand et al. 1998) was used for heterologous expression using the different expression cassettes.

Media and Culture Conditions

T. reesei strains were maintained on potato dextrose agar (Difco, Sparks, Md.) slants. Transformants were regenerated on minimum solid medium containing per liter: (NH₄)₂SO₄ 5.0 g, KH2PO₄ 15.0 g, CaCl₂ 0.45 g, MgSO₄ 0.6 g, CoCl₂ 3.7 mg, FeSO₄.H₂O 5 mg, ZnSO₄.H₂O 1.4 mg; MnSO₄.H₂O 1.6 mg, glucose as carbon source, sorbitol 182 g as osmotic stabilizer and hygromycine 125 mg for the selection. Plates were solidified and colony growth was restricted by adding 2% agar 0.1% Triton X-100 to the medium. Transformed protoplasts were plated in 3% selective top agar containing IM sorbitol.

In order to screen the FAEA activity from different transformants, fungi were grown on minimum medium containing per liter: (NH₄)₂SO₄ 5.0 g, KH₂PO₄ 15.0 g, CaCl₂ 0.6 g, MgSO₄0.6 g, CoCl₂ 3.7 mg, FeSO₄.H₂O 5 mg, ZnSO₄.H₂O 1.4 mg; MnSO₄.H₂O 1.6 mg, peptone 5 g and lactose 40 g and Solka floc cellulose (International Fiber Corporation, North Tonawanda, N.Y.) 20 g as carbon sources and inducers, Pipes 33 g to adjust pH to 5.2 with KOH. The culture medium was inoculated with 1×10⁷ spores per 50 ml and grown in conical flasks at 30° C. with shaking at 200 rpm.

Expression Vectors and Fungal Transformation

The cDNA encoding FAEA and SWOI were PCR amplified from plasmid pF (Record et al. 2003) and pMS89 including the signal peptide was amplified by using
either the F1 forward primer 5′-GATACCGCGGATGAAGCAATTCTCTGC-3′ (with the SacII site underlined)
or the F2 primer 5′-GTGCAGTTTAGCAATGCCTCCACGCAAGGCATC-3′ and the R1 reverse primer
   5′-AATACATATGTGGAGTGGTGGTGGTGGTGGTGCOAAGTACAAGCTCCGCTCG-3′ (with the NdeI site underlined, His-tag is dot lined).

The first primer pair (F1/R1) was used to obtain an amplified DNA fragment that will be used in the expression cassette pFaeA for the faeA-encoding gene (SEQ ID NO: 9) expression (Y09330) in T. reesei. The second construct was obtained by fusing the faeA gene to the gene (AJ245918) encoding SWO1 (SEQ ID NO: 1) by using an overlap extension PCR (Ho et al. 1989). In a first PCR experiment, the faeA gene was amplified by using the primer pair F2/R1 and the F3 forward primer

5′-ATATCCGCGGATGGCTGGTAAGCTTATC-3′
(with the SacII site underlined)

and the R2 reverse primer

5′-GATGCCTTGCGTGGAGGCATTCTGGCTAAACTGCAC-3′.

Both resulting overlapping fragments were mixed and a fused fragment was synthesized by using only external primers. This newly obtained fragment was cloned in the expression cassette to express the Swo1-FaeA fusion gene (pSwo-Faea).

Both amplified fragment was checked by sequencing, then ligated in the expression vector pANM1110 (cloning sites, SacII and NdeI) after digestion with SacII and NdeI restriction enzymes. In this vector, the T. reesei cellobiohydrolase I-encoding gene (cbhl) promoter was used to drive the expression of both inserts. In the first (pFaeA) and second (pSwo-FaeA) expression cassettes, the signal peptide of FAEA and SWO1, respectively, were used to initiate the secretion of the recombinant proteins.

Fungal transformation was carried out as described previously (Penttilä et al. 1987) by using the expression vectors. Transformants were purified by selection of conidia on selective medium.

Screening of the Feruloyl Esterase Activity

Cultures were monitored for 10 days at 30° C. in a shaker incubator and the pH was adjusted to 5.5 daily with a 1 M KOH. Each culture condition was performed in duplicate. From liquid culture medium, aliquots (1 mL) were collected daily and mycelia were removed by filtration. Esterase activity was assayed as previously described using methyl ferulate (MFA) as the substrate (Ralet et al. 1994). Activities were expressed in nkatal (nkat), 1 nkat being defined as the amount of enzyme that catalyzes the release of 1 nmol of ferulic acids per sec under established conditions. Each experiment was done in duplicate and measurements in triplicate. The standard deviation was recorded to less than 2% for the mean.

Protein and Western Blot Analysis

Protein concentration was determined according to Lowry et al. (1951) with bovine serum albumin as standard. Protein purification was followed by SDS-polyacrylamide gel electrophoresis on 10% polyacrylamide slab gels (Laemli 1970). Then, proteins were stained with Coomassie blue. The N-terminal sequence was determined from an electroblotted FAEA sample (40 μg) onto a poly(vinylidine difluoride) membrane (Millipore, Saint-Quentin-Yvelines, France) according to Edman degradation. Analyses were carried out on an Applied Biosystem 470A.

For Western blot analysis, total and purified proteins were electrophoresed in 11% SDS/polyacrylamide gel and electroblotted onto BA8S nitrocellulose membranes (Schleicher and Schuell, Dassel, Germany) at room temperature for 45 min. Membranes were incubated in blocking solution (50 mM Tris, 150 mM NaCl and 2% (v/v) milk pH 7.5) overnight at 4° C. Then, membranes were washed with TBS-0.2% Tween and treated with blocking solution containing anti-FAEA serum at a dilution of 1/6000. For anti-FAEA antibodies, membranes were subsequently incubated with goat anti-rabbit immunoglobin G conjugated with alkaline phosphatase (1/2500) (Promega). Alkaline phosphatase was color developed using the 5-bromo-4-chloro-3-indoyl phosphate-nitro blue tetrazolium assay (Roche Applied Science, Meylan, France) according to the manufacturer's procedure.

Purification and Characterization of the Proteins

To purity both recombinant proteins, the best isolate for each construct was inoculated in the same conditions as the screening procedure. Culture was harvested after 8 days of growth, filtered (0.7 μm) and concentrated by ultrafiltration through a polyethersulfone membrane (molecular mass cut-off of 30 kDa) (Millipore). Concentrated fractions were dialyzed against a 30 mM Tris-HCl, pH 7.0, binding buffer and the purification of His-tagged proteins was performed on a Chelating Sepharose Fast Flow column (13×15 cm) (Amersham Biosciences) (Porath et al. 1975).

The main enzymatic characteristics were determined for both recombinant proteins. Thermostability of the purified proteins (100% refers to 4.3 and 0.2 nanokatals ml⁻¹ of FAEA and SWOI-FAEA, respectively) was tested in the range of 30 to 70° C. Aliquots were preincubated at the designated temperature for 60 min and after cooling at 0° C., esterase activities was then assayed as previously indicated in standard conditions. Samples were analyzed by SDS-PAGE after incubation in order to verify integrity of the recombinant proteins. Effect of the pH on protein stability was also studied by incubating for 60 min the purified recombinant proteins in citrate-phosphate buffer (pH 2.5-7.0) and sodium phosphate (pH 7.0-8.0). All incubations were performed for 90 min, and then aliquots were transferred in standard rectional mixture to determine the amount of remaining activity. The activity determined prior to the preincubations was taken as 100% (4.3 and 0.2 nanokatals ml⁻¹ of FAEA and FAEA-SWO, respectively).

To determine optimal temperature under the conditions used, aliquots of purified recombinant proteins (100% refers to 4.3 and 0.2 nanokatals ml⁻¹ of FAEA and SWOI-FAEA, respectively) were incubated at various temperatures (30 to 70° C.) and esterase activities were assayed. Optimal pH was determined by using citrate-phosphate buffer (pH 2.5-7.0) and sodium phosphate buffer (pH 7.0-8.0) using standard-conditions.

Southern Blot Analysis

Genomic DNA of each transformants (10 μg) was digested overnight with various restriction enzymes and electrophoresed on a 0.5% agarose-TAE gel. The DNA was then blotted onto a Hybond N+ membrane and probed with a ³²P-labelled probed consisting of the faeA PCR amplified sequence. Hybridization was carried out in a buffer containing 0.5M sodium phosphate pH7.2, 0.0M EDTA, 7% (w/v) SDS, 2% (w/v) blocking agent (Roche Applied Science) overnight at 65° C. Post hybridization washes consisted of 2×15 min in 0.2 SSC (1×SSC is 0.15 M NaCl plus 0.015 M sodium citrate buffer pH 7.0), containing 1% SDS at 65° C. and 1×5 min in 0.2×SSC at room temperature. The blots were exposed to X-ray film (Biomax MR, Eastman Kodak Company, New York, USA).The wild-type A. niger strain BRFM 281 (Banque de Ressources Fongique de Marseille) was used as control containing one fae A gene copy.

Application Tests

Wheat bran (WB) was destarched and provided by ARD (Agro-industrie Recherche et Development, Pomacle, France). Enzymatic hydrolysis were performed in 0.1 M 3-(N-morpholino)propanesulfonic acid (MOPS) buffer containing 0.01% sodium azide at pH 6.0, in a thermostatically controlled shaking incubator (120 rpm) at 37° C. WB (180 mg) were incubated with the purified, FAEA, SWOI+FAEA and SWOI−FAEA, independently, in a final volume of 5 mL. Concerning test applications with culture medium from transformants, the final volume was increased to 9 ml. The enzyme concentrations were of 1.8 nkatal of esterase activity per 180 mg of dry bran for each assay. Each assay was done in duplicate and the standard deviation was less than 5% from the mean of the value for WB.

To estimate the hydroxycinnamic acid content, total alkali-extractable of phenolic compounds was determined by adding 20 mg of WB or MB in 2 N NaOH and incubated for 30 min at 35° C. in the darkness. The pH was adjusted to 2 with 2N HCl. Phenolic acids were extracted three times with 3 mL of ether. The organic phase was transferred to a test tube and dried at 40° C. One milliliter of methanol/H₂O (50:50) (v/v) was added to dry extract and samples were injected on an HPLC system as described in the next section. The total alkali-extractable ferulic acid content was considered as 100% for the enzymatic hydrolysis.

Finally to determine the ferulic acid content, enzymatic hydrolysates were diluted to ½ with acetic acid 5%, centrifuged at 12,000×g for 5 mm and supernatants were filtered through a 0.2 μm nylon filter (Gelman Sciences, Acrodisc 13, Ann Arbor, Mich.). Filtrates were analysed by HPLC (25 μL injected). HPLC analyses were performed at 280 nm and 30° C. on a HP11100 model (Hewlett-Packard Rockville, Md.) equipped with a variable UVNIS detector, a 100-position autosampler-autoinjector. Separations were achieved on a Merck RP-18 reversed-phase column (Chromolith 3.5 μm, 4.6×100 mm, Merck). The flow rate was 1.4 mL/min. The mobile phase used was 1% acetic acid and 10% acetonitrile in water (A) versus acetonitrile 100% (B) for a total running time of 20 min, and the gradient changed as follows: solvent B started at 0% for 2 min, then increased to 50% in 10 min, to 100% in 3 min until the end of running. Data were processed by a HP 3365 ChemStation and quantification was performed by external standard calibration.

### Example 1

### Fungal Transformation and Production of the Recombinant Enzymes

Two T. reesei strains, Rut-C30 and CL847, used by industrial companies to produce in controlled fermentation processes large amount of enzymes, were transformed by expression vectors containing genes of interest. In a first construct, the faeA gene from A. niger was placed under the control of the cbhI gene promoter using the signal peptide of the FAEA to target the secretion. The recombinant FAEA was used in the following application tests as a control. In a second construct, the faeA gene was fused to the swoI-encoding gene to produced a chimerical protein associating the A. niger FAEA to the T. reesei SWOI protein. In this construct, the signal peptide of SWOI was used for secretion of the recombinant protein. Protoplastes obtained from both strains were transformed independently by both genetic cassettes cloned in the expression vector pAMH10. Transformants were then selected for their abilities to grow in minimal medium containing hygromycine. Approximately three hundred transformants were filter purified by selection of conidia on selective medium, and more or less 150 hygromycine-resistant colonies were screened by detecting the feruloyl esterase activity produced in the culture medium, and by performing a western blot analysis. Considering all the transformants, only a feriloyl activity was detectable for those transformed by pFaeA (FaeA transformants). In addition, the production of FAEA was confirmed by western blot analysis. Concerning T. reesei colonies transformed by pSwo-FaeA (Swo-FaeA transformants), a FAEA production was only detected by western blot analysis, because the feruloyl esterase activity was very low produced. However, for the FaeA transformants, 1.3 and 23.3% of the colonies were shown to produce a feruloyl esterase activity, respectively, for Rut-C30 and CL847 strains. In the second transformation event using pSwo-FaeA, the percentage was higher, with 23.3 and 51.7% of the colonies, respectively. For each construct and T. reesei strains, the best producing transformants was then cultured to study the time course of the feruloyl esterase activity.

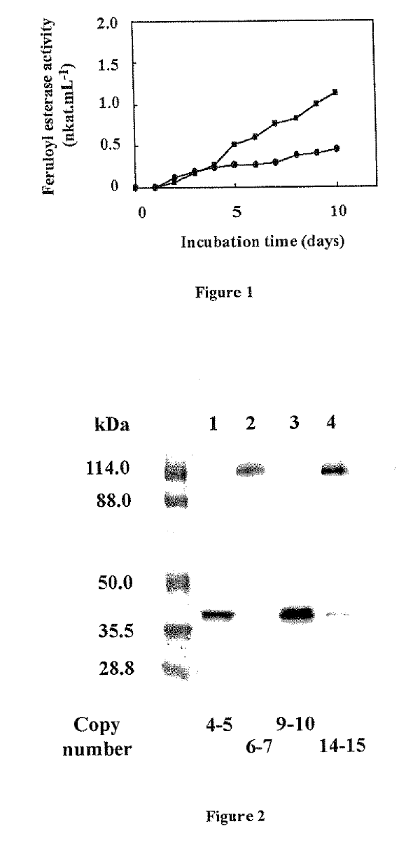

Esterase activity was estimated in both transformed Rut-C30 and CL847 that were transformed by pFAEA and reported as a function of time (FIG. 1). In both cases, esterase activity was detectable already on day 2 and increased progressively to 0.45 and 1.15 nkatal mL⁻¹, respectively. Concerning T. reesei transformed by pSwo-FaeA, a low activity was measured on day 8 of approximately 0.06 nkatal mL⁻¹ for both strains Western blot analysis were performed from the culture medium of FaeA transformants (FIG. 2) and a band of approximately 40 kDa corresponding to the recombinant FAEA was showed (FIG. 2, lanes 1 and 3). Beside this first set of fungal transformants, the Swo-FaeA transformants produced a major band of approximately 120 kDa corresponding to the fusion of the FAEA (36 kDa) and the SWOI protein (75 kDa) (FIG. 2, lane 2 and 4). Furthermore, a weak band of 40 kDa appeared that corresponds to the size of the FAEA. Finally, the copy number of expression cassettes integrated in the fungal genome was estimated by Southern blot analysis and revealed that the FaeA transformants contains 4 to 5 and 9 to 10 copies, respectively for strains Rut-C30 and CL847. Concerning the Swo-FaeA transformant set, 6 to 7 and 14 to 15 copies were estimated for both strains, respectively. As the T. reesei CL847 has produces the same amount of SWOI-FAEA than the Rut-C30 strain, but higher yield of FAEA, the following experiments were performed with proteins obtained from this strain.

### Example 2

### Characterization of the Recombinant Enzymes

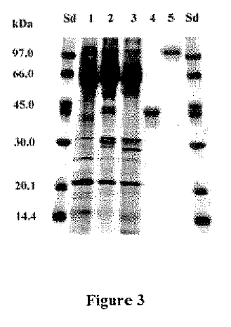

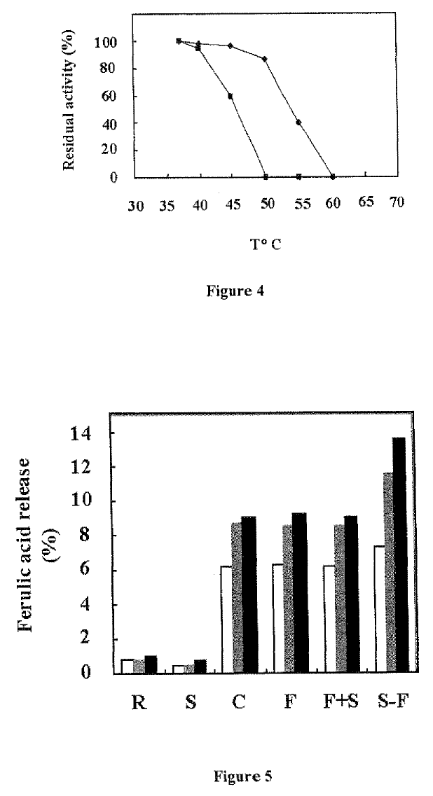

The purified FAEA and chimerical SWOI-FAEA were purified on a Chelating Sepharose column and the homogeneity of proteins was checked on an SDS/polyacrylamide gel (FIG. 3). The molecular mass of the recombinant FAEA were slightly higher than expected as compared to the FAEA produced in A. niger. Both N-terminal sequences of the FAEA (ASTQG) and the SWOI-FAEA (QQNCA) were sequenced and were found to be 100% identical to those of the corresponding native proteins, demonstrating that the processing was correct. All the main physico-chemical and kinetic properties were further determined and compared to the FAEA from A. niger (Record et al. 2003) (Table I and FIG. 4). Considering the effect of temperature and pH, as well as the pH stability, no significant difference was found. The temperature stability of both proteins were also estimated and our results showed that the recombinant FAEA was stable until 45° C. and that the activity decreased by 60% after an incubation of 60 min at 55° C. No remaining activity was found at 60° C. On the other hand, concerning the SWOI-FAEA protein, activity was stable until 40° C. and no remaining activity was detected after a 60-min incubation at 50° C. No great difference was found for the Km value. But, while Vm and specific activities were in the same range for the FAEA produced by A. niger and T. reesei, a clear shift was observed for the SWOI-FAEA protein that was found to be less efficient to hydrolyse the methyl ferulate that the corresponding FAEA.

### Example 3

### Enzymatic Release of Ferulic Acid from Wheat Bran

The synergistic effect generated by the physical proximity of the FAEA and SWOI was studied for the release of ferulic acid from wheat bran. Wheat bran was incubated with purified enzymes (FIG. 5) and results showed that FAEA produced in A. niger and T. reesei was able to release the same amount of ferulic acid, i.e. from 6 to 9% depending on the incubation time. Considering the SWOI, the native protein alone or in addition with FAEA (S or F+S) was not efficient if compared to the reference or the experiment with FAEA, respectively. On the other hand, a significant higher value of ferulic acid release, i.e. from 7 to 13.5% was obtained with the SWOI-FAEA protein corresponding to an improvement factor of 1.5 after 24 hour of hydrolysis

The recombinant CL847 strain producing the recombinant SWOI-FAEA was evaluated for the release of ferulic acid using the total extracellular cocktail of secreted enzymes. While the extracellular medium obtained form the non transformed parental strain was able to release 0.5 to 1.8% of ferulic acid from 4 to 24 hours of incubation, the transformed CL847 strain secreted an enzymatic cocktail including the SWOI-FAEA that released up to 45% until 4 hours, i.e. 1.8 g of ferulic acid by kg of wheat bran. This yield did not increase even after 24 hours of incubation.

TABLE 1
Physico-chemical and kinetic characteristics
of the recombinant feruloyl esterase and the
chimerical enzyme from Trichoderma reesei

| | FAEA^{a} | FAEA | SWOI-FAEA |
|---|---|---|---|
| MM (kDa) | 36 | 40 | 120 |
| Tp optimum (° C.) | 55 | 50-55 | 50 |
| Tp stability (° C.) | — | 45 | 40 |
| pH optimum | 5 | 5 | 5 |
| pH stability | 5-6 | 5-6 | 5-6 |
| Km^{b} | 0.75 | 0.83 | 0.81 |
| Vm^{c} | 382 | 291 | 52 |
| Specific activity^{d} | 20 | 16.4 | 2.6 |

^{a}estimated from the Aspergillus niger feruloyl esterase (Record et al. 2003)
^{b}Km were expressed in millimolar
^{c}Vm were expressed in nanokatal per mg of protein
^{d}Specific activities were expressed in nanokatal per mg of protein

^{c}Vm were expressed in nanokatal per mg of protein
^{d} Specific activities were expressed in nanokatal per mg of protein

Activities were assayed using methyl ferulate as substrate

### REFERENCES

Boraston A B, Bolarn D N, Gilbert H J, Davies G J (2004) Carbohydrate-binding modules: fine-tuning polysaccharide recognition. Biochem J, 382: 769-781.
Ciruela A H J, Gilbert B R Ali, Hazlewood G P (1998) Synergistic interaction of the cellulosome integrating protein (CipA) from Clostridium thermocellum with a cellulosomal endoglucanase. FEBS Lett 422:221-224,
Cosgrove D J (2000) Expansive growth of plant cell walls. Plant Physiol Biochem. 38:109-24
Crepin V F, Faulds C B, Connerton I F (2003) Functional classification of the microbial feruloyl esterases. Appl Microbiol Biotechnol 63:647-52.
Cosgrove D J (2000) Loosening of plant cell wall by expansin, Nature 407:321-326.
de Vries R P, Michelsen B, Poulsen C H., Kroon Pa., van den Heuvel R H H, Faulds C B, Williamson G, van den Hombergh J P T W, Visser J (1997). The faeA genes from Aspergillus niger and Aspergllus tubingensis encode ferulic acid esterases involved in degradation of complex cell wall polysaccharides. Appl Environ Microbiol 63:4638-4644.
de Vries R P, VanKuyk P A, Kester H C M, Visser. J. (2002) The Aspergillus niger faeb gene encodes a second feruloyl esterase involved in pectin and xylan degradation and is specifically induced in the presence of aromatic compounds. Biochem J 363, 377-386.
Din N, Gilkes N R, Tekan B, Miller R C Jr, Warren R A J, Kilburn D G (1991) Non-hydrolytic disruption of cellulose fibres by the binding domain of a bacterial cellulose. Bio/technol 9-1096-1099.
Durand H, Baron M, Calmels T, Tiraby G (1988) Classical and molecular genetics applied to Trichoderma reesei for the selection of improved cellulolytic industrial strains, FEMS Symp 43:1135-152.
Faulds C B, Williamson G (1994) Purification and characterization of a ferulic acid esterase (FAEIII) from Aspergillus niger: specificity for the phenolic moiety and binding to microcrystalline cellulose. Microbiology 140:779-787.
Fierobe, H. P., E. A Bayer, C. Tardif M. Czjzek, A. Mechaly, A. Belaich, R. Lamed, Y. Shoham, and J. P. Belaich. 2002. Degradation of cellulose substrates by cellulosome chimeras. Substrate targeting versus proximity of enzyme components. J. Biol. Chem. 277:49621-49630.
Gao P J, Chen G J, Wang T H, Zhang Y S, Liu J (2001) Non-hydrolytic disruption of crystalline structure of cellulose by cellulose binding domain and linker sequence of cellobiohydrolase I from Penicillium janthinellum. Shengwu Huaxue Yu Shengwu Wuli Xuebao 33, 13-18.
Ho, S. N., H. D. Hunt, R. M Horton, J. K. Pullen, L. R. Pease. 1989. Site-directed mutagenesis by overlap extension using the polymerase chain reaction. Gene. 77:51-59.
Ito J, Fujita Y, Ueda M, Fukuda H, Kondo A (2004) Improvement of cellulose-degrading ability of a yeast strain displaying Trichoderma reesei endoglucanase II by recombination of cellulose-binding domains. Biotechnol Prog 20:688-91.
Juge N, Wiliamson G, Puigserver A, Cummings N J, Connerton I F, Faulds C B (2001) High-level production of recombinant Aspergillus niger cinnamoyl esterase (FAEA) in the methylotrophic yeast Pichia pastoris. FEMS Yeast Research 1:127-132.
Kroon, P. A., Faulds C. B., Williamson G (1996) Purification and characterization of a novel esterase induced by growth of Aspergillus niger on sugar beet pulp. Biotechlol. Appl. Biochem. 23, 255-262.
Laemli U K (1970) Cleavage of structural proteins during the assembly of the head of bacteriophage T4. Nature 227:680-685.
Levasseur A, Benoit 1, Asther M, Asther M, Record E (2004) Homologous expression of the feruloyl esterase B gene from Aspergillus niger and characterization of the recombinant enzyme. Protein Expr Purif 37:126-33.
Levasseur A., Navarro D., Punt P. J., Belaich J-P., Asther M., Record E. (2005) Improvement of ferulic acid release by action of engineered bifunctional enzymes associating the feruloyl esterase A, the xylanase B and a carbohydrate-binding module from Aspergillus niger. Appl and Envir Microbiol, 71:8132-8140.
Li Y, Jones L, McQueen-Mason (2003) Expansins and cell growth. Curr Opinion Plant Biol 6:603-610.
Limon M C, Chacon M R, Mejias R, Delgado-Jarana J, Rincon A M, Codon A C, Benitez T (2004) Increased antifingal and chitinase specific activities of Trichoderma harzianum CECT 2413 by addition of a cellulose binding domain. Appl Microbiol Biotechnol 64:675-85.
Lowry O H, Rosebrough N J, Farr A L, Randall R J (1951) Protein measurement with the Folin phenol reagent. J Biol Chem 193:265-275.
Montenecourt B S, Eveleigh D E (1977) Selective screening methods for the isolation of high yielding cellulase mutants of Trichoderma reesei on various carbon sources. Adv Chem Ser 181:289-301.
Penttila M, Nevalainen H, Ratto M, Salminen E, Knowles J. (1987) A versatle transformation system for the cellulolytic filamentous fungus Trichoderma reesei. Gene 61:155-64.
Porath, J, Carlsson J, Olsson I, Belfrage G (1975). Metal chelate affinity chromatography, a new approach to protein fractionation. Nature. 258:598-599.
Record, E., Asther M., Sigoillot C., Pages S., Punt P. J., Delattre M., Haon M., van den Hondel C. A., Sigoillot J. C., Lesage-Meessen L., Asther M. (2003) Overproduction of the Aspergillus niger feruloyl esterase for pulp bleaching application. Appl. Microbiol. Biotechnol. 62:349-355.
Ralet M. C, Faulds C B, Williamson G, Thibault J F (1994) Degradation of feruloylated oligosaccharides from sugar-beet pulp and wheat bran by ferulic acid esterases from Aspergillus niger. Carbohydr Res 263:257-269.
Reese E T, Siu R G H, Levinson H S (1950) The biological degradation of soluble cellulose derivatives and its relationship to the mechanism of cellulose hydrolysis, J Bacteriol 59, 485-497.
Saloheimo M., Paloheimo M., Hakola S., Pere J., Swanson B., Nyyssonen E., Bhatia A., Ward M., Penttila M. (2002). Swollenin, a Trichoderman reesei protein with sequence similarity to the plant expansins, exhibits disruption activity on cellulosic materials. Eur J Biochem 269:4202-4211.
Saulnier L, Thibault J F (1999) Ferulic acid and diferulic acids as components of sugar-beet pectins and maize bran heteroxylans. J Sci Food Agric 79:396-402.
Sigoillot C, Camarero S Vidal T, Record E, Asther M, Perez-Boada M, Martinez M J, Sigoillot J C, Asther M, Colom J F, Martinez A T (2005) Comparison of different fungal enzymes for bleaching high-quality paper pulps. J Biotechnol 115:333-43.
Williamson G, Kroon P A, Faulds G B (1998) Hairy plant polysaccharides: a close shave with microbial esterases. Microbiol. 144:2011-2023.

1. Fusion proteins comprising:
at least a swollenin, i.e. a protein containing a carbohydrate-binding-molecule (CBM) domain which targets the cellulose of plants, and an expansin domain which breakdowns hydrogen bounds between cellulose microfibrils,
and at least a plant cell-wall degrading enzyme, said enzyme being such that it contains a CBM domain or not, provided that when it contains a CBM this latter may be deleted if necessary,
said swollenin, and plant cell-wall degrading enzyme, being recombinant proteins corresponding to native proteins in fungi, or mutated forms thereof.

2. Fusion proteins according to claim 1, wherein the swollenin corresponds to native proteins, or mutated forms thereof, from fungi chosen among ascomycetes, such as:
Trichoderma strains, and more particularly Trichoderma reesei, or Aspergillus strains, and more particularly Aspergillus fumigatus.

3. Fusion proteins according to claim 1, wherein the swollenin corresponds to native enzymes, or mutated forms thereof, from Trichoderma strains, such as Trichoderma reesei.

4. Fusion proteins according to claim 1, wherein the swollenin is the protein of Trichoderma reesei, represented by SEQ ID NO: 2 with its signal peptide, or by SEQ ID NO: 4 in its mature state.

5. Fusion proteins according to claim 1, wherein the swollenin corresponds to native enzymes, or mutated forms thereof, from Aspergillus strains, such as Aspergillus fumigatus.

6. Fusion proteins according to claim 5, wherein the swollenin is the protein of Aspergillus fumigatus, represented by SEQ ID NO: 6 with its signal peptide, or by SEQ ID NO: 8 in its mature state.

7. Fusion proteins according to claim 1, wherein the plant cell-wall degrading enzymes are chosen among enzymes able to hydrolyze cellulose, hemicellulose, and degrade lignin.

8. Fusion proteins according to claim 1, wherein the plant cell-wall degrading enzymes are hydrolases chosen among:
cellulases, such as endoglucanases, exoglucanases such as cellobiohydrolases, or β-glucosidases,
hemicellulases, such as xylanases,
ligninases able to degrade lignins, such as laccases, manganese peroxidase, lignin peroxidase, versatile peroxidase, or accessory enzymes such as cellobiose deshydrogenases, and aryl alcohol oxidases,
cinnamoyl ester hydrolases able to release cinnamic acids such as ferulic acids and to hydrolyse diferulic acid cross-links between hemicellulose chains, such as feruloyl esterases, cinnamoyl esterases, and chlorogenic acid hydrolases.

9. Fusion proteins according to claim 1, wherein the plant cell-wall degrading enzymes are chosen among feruloyl esterases, cellobiohydrolases with or without their CBM domains, endoglucanases with or without their CBM domains, xylanases, and laccases.

10. Fusion proteins according to claim 1, wherein the plant cell-wall degrading enzymes correspond to native enzymes, or mutated forms thereof, from fungi chosen among:
ascomycetes, such as:
Aspergillus strains, and more particularly Aspergillus niger,
Trichoderma strains, and more particularly Trichoderma reesei,
Magnaporthe strains, and more particularly Magnaporthe grisea,
basidiomycetes, such as Pycnoporus, Halocyphina, or Phanerochaete strains, and more particularly Pycnoporus cinnabarinus, Pycnoporus sanguineus, or Halocyphina villosa, or Phanerochaete chrysosporium.

11. Fusion proteins according to claim 1, wherein the plant cell-wall degrading enzymes correspond to native enzymes, or mutated forms thereof, from Aspergillus strains, such as Aspergillus niger.

12. Fusion proteins according to claim 1, wherein at least one of the plant cell-wall degrading enzymes is a feruloyl esterase, such as the one chosen among:
the feruloyl esterase A of A. niger represented by SEQ ID NO: 10,
or the feruloyl esterase B of A. niger represented by SEQ ID NO: 12.

13. Fusion proteins according to claim 1, wherein at least one of the plant cell-wall degrading enzymes is a xylanase such as the xylanase B of A. niger represented by SEQ ID NO: 14.

14. Fusion proteins according to claim 1, wherein the plant cell-wall degrading enzymes correspond to native enzymes, or mutated forms thereof, from Trichoderma strains, such as Trichoderma reesei.

15. Fusion proteins according to claim 14, wherein at least one of the plant cell-wall degrading enzymes is a cellobiohydrolase, such as the one chosen among:
the cellobiohydrolase I of T. reesei, and represented by SEQ ID NO: 16,
the cellobiohydrolase I of T. reesei, wherein the CBM domain has been deleted, and represented by SEQ ID NO: 18,
the cellobiohydrolase II of T. reesei, and represented by SEQ ID NO: 20,
the cellobiohydrolase II of T. reesei, wherein the CBM domain has been deleted, and represented by SEQ ID NO: 22.

16. Fusion proteins according to claim 14, wherein at least one of the plant cell-wall degrading enzymes is an endoglucanase, such as the one chosen among:
the endoglucanase I of T. reesei, and represented by SEQ ID NO: 24,
the endoglucanase I of T. reesei, wherein the CBM domain has been deleted, and represented by SEQ ID NO: 26.

17. Fusion proteins according to claim 1, comprising linkers between at least two of the proteins comprised in said fusion proteins, said linkers being polypeptides from 10 to 100 aminoacids, advantageously of about 50 aminoacids.

18. Fusion proteins according to claim 1, wherein a linker is included between each protein comprised in said fusion proteins.

19. Fusion proteins according to claim 1, wherein the linker is a hyperglycosylated polypeptide such as the sequence represented by SEQ ID NO: 28, present in the cellobiohydrolase B of A. niger.

20. Fusion proteins according to claim 1, chosen among the fusion proteins of the swollenin of Trichoderma reesei represented by SEQ ID NO: 4, with:
the feruloyl esterase A of A. niger represented by SEQ ID NO: 10, said fusion protein being represented by SEQ ID NO: 30,
the feruloyl esterase A of A. niger represented by SEQ ID NO: 10, said fusion protein comprising the sequence represented by SEQ ID NO: 28 as a hyperglycosylated linker between SEQ ID NO: 4 and SEQ ID NO: 10, and being represented by SEQ ID NO: 32,
the feruloyl esterase B of A. niger represented by SEQ ID NO: 12, said fusion protein being represented by SEQ ID NO: 34, the feruloyl esterase B of A. niger represented by SEQ ID NO: 12, said fusion protein comprising the sequence represented by SEQ ID NO: 28 as a hyperglycosylated linker between or SEQ ID NO: 4 and SEQ ID NO: 12, and being represented by SEQ ID NO: 36,
the-xylanase B of A. niger represented by SEQ ID NO: 14, said fusion protein being represented by SEQ ID NO: 38,
the xylanase B of A. niger represented by SEQ ID NO: 14, said fusion protein comprising the sequence represented by SEQ ID NO: 28 as a hyperglycosylated linker between SEQ ID NO: 4 and SEQ ID NO: 14, and being represented by SEQ ID NO: 40,
the cellobiohydrolase I of T. reesei represented by SEQ ID NO: 16, said fusion protein being represented by SEQ ID NO: 42,
the cellobiohydrolase I of T. reesei represented by SEQ ID NO: 16, said fusion protein comprising the sequence represented by SEQ ID NO: 28 as a hyperglycosylated linker between SEQ ID NO: 4 and SEQ ID NO: 16, and being represented by SEQ ID NO: 44,
the cellobiohydrolase I of T. reesei without its endogenous CBM represented by SEQ ID NO: 18, said fusion protein being represented by SEQ ID NO: 46,
the cellobiohydrolase I of T. reesei without its endogenous CBM represented by SEQ ID NO: 18, said fusion protein comprising the sequence represented by SEQ ID NO: 28 as a hyperglycosylated linker between SEQ ID NO: 4 and SEQ ID NO: 18, and being represented by SEQ ID NO: 48,
the cellobiohydrolase II of T. reesei by SEQ ID NO: 20, said fusion protein being represented by SEQ ID NO: 50,
the cellobiohydrolase II of T. reesei represented by SEQ ID NO: 20, said fusion protein comprising the sequence represented by SEQ ID NO: 28 as a hyperglycosylated linker between SEQ ID NO: 4 and SEQ ID NO: 20, and being represented by SEQ ID NO: 52,
the cellobiohydrolase II of T. reesei without its endogenous CBM represented by SEQ ID NO: 22, said fusion protein being represented by SEQ ID NO: 54,
the cellobiohydrolase II of T. reesei without its endogenous CBM represented by SEQ ID NO: 22, said fusion protein comprising the sequence represented by SEQ ID NO: 28 as a hyperglycosylated linker between SEQ ID NO: 4 and SEQ ID NO: 22, and being represented by SEQ ID NO: 56,
the endoglucanase I of T. reesei represented by SEQ ID NO: 24, said fusion protein being represented by SEQ ID NO: 58,
the endoglucanase I of T. reesei represented by SEQ ID NO: 24, said fusion protein comprising the sequence represented by SEQ ID NO: 28 as a hyperglycosylated linker between SEQ ID NO: 4 and SEQ ID NO: 24, and being represented by SEQ ID NO: 60,
the endoglucanase I of T. reesei without its endogenous CBM represented by SEQ ID NO: 26, said fusion protein being represented by SEQ ID NO: 62,
the endoglucanase I of T. reesei without its endogenous CBM represented by SEQ ID NO: 26, said fusion protein comprising the sequence represented by SEQ ID NO: 28 as a hyperglycosylated linker between SEQ ID NO: 4 and SEQ ID NO: 26, and being represented by SEQ ID NO: 64.

21. Fusion proteins according to claim 1, chosen among the fusion proteins of the swollenin of Aspergillus fumigatus represented by SEQ ID NO: 8, with:
the feruloyl esterase A of A. niger represented by SEQ ID NO: 10, said fusion protein being represented by SEQ ID NO: 66,
the feruloyl esterase A of A. niger represented by
SEQ ID NO: 10, said fusion protein comprising the sequence represented by SEQ ID No: 28 as a hyperglycosylated linker between SEQ ID NO: 8 and SEQ ID NO: 10, and being represented by SEQ ID NO: 68,
the feruloyl esterase B of A. niger represented by SEQ ID NO: 12, said fusion protein being represented by SEQ ID NO: 70,
the feruloyl esterase B of A. niger represented by
SEQ ID NO: 12, said fusion protein comprising the sequence represented by SEQ ID NO: 28 as a hyperglycosylated linker between or SEQ ID NO: 8 and SEQ ID NO: 12, and being represented by SEQ ID NO: 72,
the-xylanase B of A. niger represented by SEQ ID NO: 14, said fusion protein being represented by SEQ ID NO: 74,
the xylanase B of A. niger represented by SEQ ID NO: 14, said fusion protein comprising the sequence represented by SEQ ID NO: 28 as a hyperglycosylated linker between SEQ ID NO: 8 and SEQ ID NO: 14, and being represented by SEQ ID NO: 76,
the cellobiohydrolase I of T. reesei represented by SEQ ID NO: 16, said fusion protein being represented by SEQ ID NO: 78,
the cellobiohydrolase I of T. reesei represented by SEQ ID NO: 16, said fusion protein comprising the sequence represented by SEQ ID NO: 28 as a hyperglycosylated linker between SEQ ID NO: 8 and SEQ ID NO: 16, and being represented by SEQ ID NO: 80,
the cellobiohydrolase I of T. reesei without its endogenous CBM represented by SEQ ID NO: 18, said fusion protein being represented by SEQ ID NO: 82,
the cellobiohydrolase I of T. reesei without its endogenous CBM represented by SEQ ID NO: 18, said fusion protein comprising the sequence represented by SEQ ID NO: 28 as a hyperglycosylated linker between SEQ ID NO: 8 and SEQ ID NO: 18, and being represented by SEQ ID NO: 84,
the cellobiohydrolase II of T. reesei by SEQ ID NO: 20, said fusion protein being represented by SEQ ID NO: 86,
the cellobiohydrolase II of T. reesei represented by SEQ ID NO: 20, said fusion protein comprising the sequence represented by SEQ ID NO: 28 as a hyperglycosylated linker between SEQ ID NO: 8 and SEQ ID NO: 20, and being represented by SEQ ID NO: 88,
the cellobiohydrolase II of T. reesei without its endogenous CBM represented by SEQ ID NO: 22, said fusion protein being represented by SEQ ID NO: 90,
the cellobiohydrolase II of T. reesei without its endogenous CBM represented by SEQ ID NO: 22, said fusion protein comprising the sequence represented by SEQ ID NO: 28 as a hyperglycosylated linker between SEQ ID NO: 8 and SEQ ID NO: 22, and being represented by SEQ ID NO: 92,
the endoglucanase I of T. reesei represented by SEQ ID NO: 24, said fusion protein being represented by SEQ ID NO: 94,
the endoglucanase I of T. reesei represented by SEQ ID NO: 24, said fusion protein comprising the sequence represented by SEQ ID NO: 28 as a hyperglycosylated linker between SEQ ID NO: 8 and SEQ ID NO: 24, and being represented by SEQ ID NO: 96,
the endoglucanase I of T. reesei without its endogenous CBM represented by SEQ ID NO: 26, said fusion protein being represented by SEQ ID NO: 98,
the endoglucanase I of T. reesei without its endogenous CBM represented by SEQ ID NO: 26, said fusion protein comprising the sequence represented by SEQ ID NO: 28 as a hyperglycosylated linker between SEQ ID NO: 4 and SEQ ID NO: 26, and being represented by SEQ ID NO: 100.

22. Nucleic acids encoding a fusion protein as defined in claim 1.

23. Vectors transformed with a nucleic acid as defined in claim 22.

24. Host cells transformed with a nucleic acid as defined in claim 22.

25. Transformed host cells according to claim 24, chosen among fungi cells, selected from A. niger, A. fumigatus, Trichoderma reesei, or Pycnoporus cinnabarinus.

26. Process for the preparation of fusion proteins, comprising the culture in vitro of host cells according to claim 24, the recovery, and if necessary, the purification of the fusion proteins produced by said host cells in culture.

27-28. (canceled)

29. Process of plant cell-wall degradation in the frame of the preparation, from plants or vegetal by-products, of compounds of interest located in plant cell-wall, characterized in that it comprises the following steps: the enzymatic treatment of plants or vegetal by-products or industrial waste, with fusion proteins according to claim 1,
optionally, the physical treatment of plants or vegetal by-products by steam explosion in combination with the action of fusion proteins,
optionally, the biotransformation with appropriate microorganisms or enzymes of the compounds contained in the cell walls and released during the above enzymatic treatment,
the recovery, and if necessary, the purification, of the compound of interest released from the cell walls during the above enzymatic treatment or obtained during the above biotransformation step.
